(19) United States
(12) Patent Application Publication
(10) Pub. No.: US 2012/0283338 A1
(43) Pub. Date: Nov. 8, 2012

(54) SWEETENER COMPOSITIONS AND METHODS OF MAKING THE SAME

(75) Inventors: Jessica Adkins, Kimball Township, MI (US); Martin N. Aschauer, Port Huron, MI (US); Janice Marie Johnson, Eden Prairie, MN (US); Chin Hong Paul Kim, Palisades Park, NJ (US); Michael Scott Oiler, Liberty, MO (US)

(73) Assignee: CARGILL ,INCORPORATED, Wayzata, MN (US)

(21) Appl. No.: 13/512,070

(22) PCT Filed: Nov. 24, 2010
(86) PCT No.: PCT/US2010/058058

§ 371 Date: May 25, 2012

Related U.S. Application Data
(60) Provisional application No. 61/264,459, filed on Nov. 25, 2009.

(51) Int. Cl. A23L 1/221; C11B 5/00; A61K 47/36; A21D 13/00; A23L 1/212; A23L 1/39; A23G 9/00; A23L 2/00; A23L 1/302; A23L 1/31

(52) U.S. Cl. 514/777; 426/650; 426/72; 426/541; 426/648; 426/641; 426/549; 426/615; 426/589; 426/565; 426/100; 426/590

(57) ABSTRACT

Disclosed is a method of preparing a composition by mixing a sweetener and an ingredient, pressing the sweetener and ingredient together to form a pressed mixture in the form of a sheet, briquette, pellet, flake or other form, and reducing the pressed mixture to yield a composition. The composition comprises particulates having a desirable size, where a particulate comprises the ingredient at least partially embedded within the sweetener. The composition can be used in a variety of food, dietary and pharmaceutical products.

### CROSS REFERENCE TO RELATED APPLICATION

This application claims the benefit of the U.S. Provisional Patent Application Ser. No. 61/264,459 filed Nov. 25, 2009, entitled SWEETENER COMPOSITIONS AND METHODS OF MAKING THE SAME, which is hereby incorporated by reference in its entirety.

### BACKGROUND

Sweetener compositions of multiple components are usually formed in a number of ways, many of which are energy intensive. Existing methods for mixing and preparing such compositions include processes such as heating or melting, extruding, boiling, super-saturating an aqueous solution, cooling, drying or combinations of these steps. Other methods involve the use of solvents for extraction and steps to remove such solvents. The existing processes are often energy-intensive, can result in a loss of certain ingredients through the processing steps, especially ingredients such as volatile or heat-sensitive compounds, and can result in off-flavors from the use of solvents. There exists a need for processes that produce compounds with retention of the taste and flavor of the original ingredients that consume less energy and are therefore more economical.

### SUMMARY

The present disclosure relates to a method of preparing a composition comprising mixing a sweetener and an ingredient where the sweetener comprises more than 50% of the mixture, pressing the sweetener and ingredient together under sufficient pressure to form a pressed mixture, and reducing the size of the pressed mixture to yield a composition. The composition can comprise particulates where a particulate comprises the ingredient at least partially embedded within the sweetener. The sweetener and ingredient can be pressed together by compacting, flaking, pelletizing, or briquetting them into a pressed mixture that is, for instance, a sheet, a briquette, flakes, or pellets, and subsequently reduced into particulates having a desired size. An average particulate is a composition of the sweetener and the ingredient, where the ingredient is at least partially embedded into the sweetener. A typical particulate of the composition will impart a similar taste profile, aroma and color to each portion of the food product to which it is applied. The particulates can be further separated between conforming and nonconforming sizes via a separation device. At least a portion of the nonconforming composition can be recycled back into the process in the mixing, pressing, or reducing steps.

In other embodiments, a composition is prepared wherein the composition comprises a sweetener matrix and an ingredient selected from the group consisting of a flavor, seasoning agent, coloring agent, aroma agent, masking agent, enhancing agent, high potency agent, compounded flavor, extract, spice, nutrient, micronutrient, oil, herb, mineral, inorganic salt, organic salt, salt substitute, vitamin, emulsifying agent, stabilizing agent, anti-caking agent, dietary supplement, antioxidant, or combinations thereof, wherein the ingredient is at least partially embedded within the sweetener matrix. In another embodiment, the sweetener matrix comprises a natural, artificial, nutritive or non-nutritive sweetener or combinations thereof. The sweetener can comprise sugars, sugar alcohols, monosaccharides, disaccharides, trisaccharides, oligosaccharides, sugar substitutes, or mixtures thereof. The composition may exhibit altered or enhanced physical properties such as, for example, a high solubility rate, low bulk density, and/or improved adherence to a product or substrate. The composition can be used on or in a variety of food products, dietary products, or pharmaceutical products.

These and other aspects of the present invention are elucidated further in the detailed description.

### BRIEF DESCRIPTION OF THE DRAWINGS

The present invention will become more fully understood from the detailed description given below and the accompanying drawings. These drawings are given by way of illustration only, and thus are not intended to be limiting of the present invention.

FIG. 1 is a block diagram that illustrates an example of a process for preparing a composition of a sweetener and an ingredient.

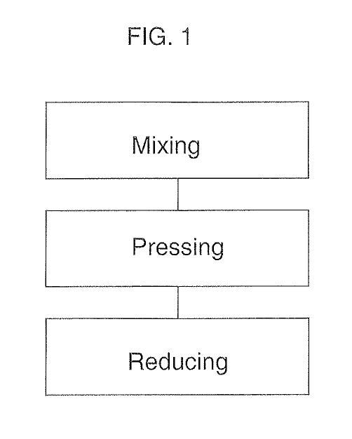

FIG. 2 is a block diagram that illustrates another example of a process for preparing a composition of a sweetener and an ingredient.

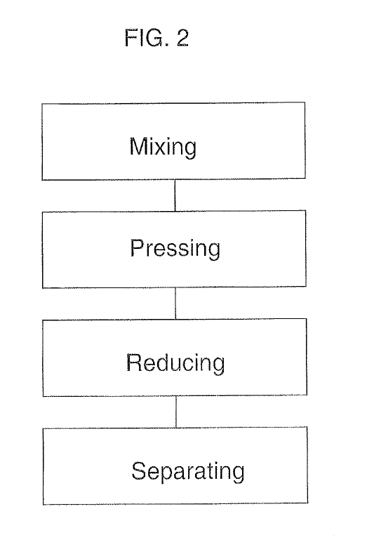

FIG. 3 is a block diagram that illustrates another example of a process for preparing a composition of a sweetener and an ingredient.

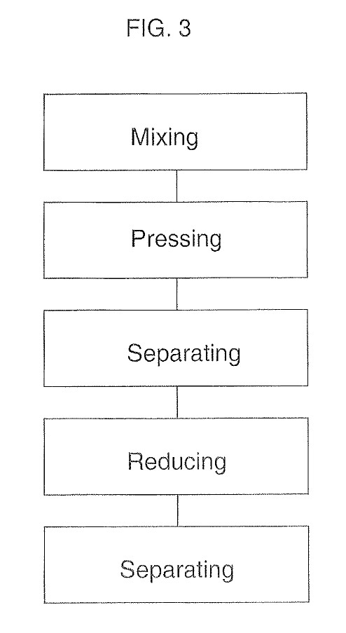

FIG. 4 is a schematic diagram that illustrates an example of a process for preparing a composition of a sweetener and an ingredient.

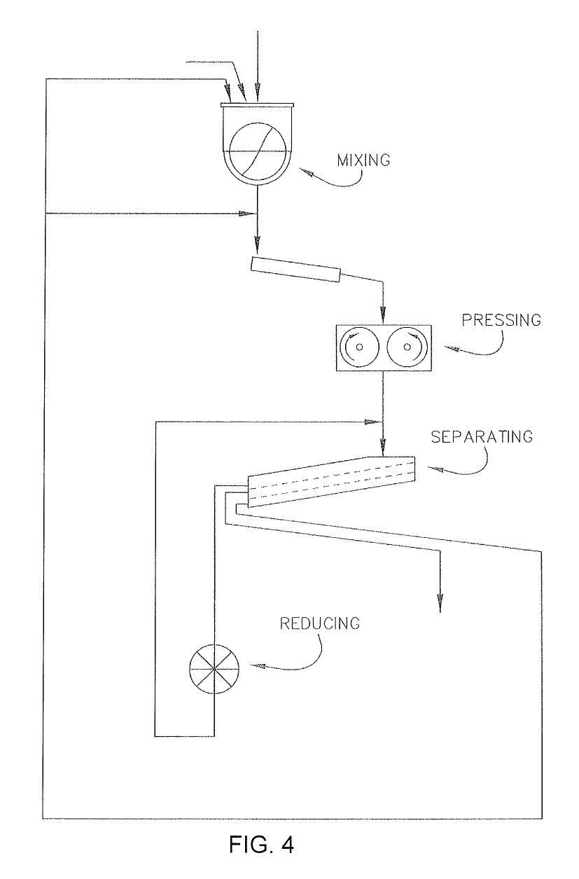

FIG. 5 is a series of four pictures, top lit and back lit, of sucrose with 2.0% orange (043-00300 Powder 8X16).

### DETAILED DESCRIPTION

### Selected Definitions

As used herein, the following terms shall have the following meanings:

The teen “sweetener” as used herein refers to any number of compounds that are used to sweeten other products. Sweeteners can include sugars, sugar alcohols, monosaccharides, disaccharides, trisaccharides, oligosaccharides, sugar substitutes, or mixtures thereof.

The term “ingredient” as used herein is a compound that is mixed with the sweetener to form a composition.

The term “mixing” as used herein is a process of physically mixing the components of the composition, specifically the sweetener and the ingredient. Mixing can occur by any number of methods.

The term “mixture” as used herein is a combination of the sweetener and the ingredient.

The term “pressing” as used herein is the step of using mechanical pressure on the mixture of the sweetener and the ingredient to form a pressed mixture. The pressing step can occur by any number of methods, including by compaction, flaking, pelletizing or briquetting.

The term “pressed mixture” as used herein is the compound resulting from pressing the sweetener and ingredient together.

The term “reducing” as used herein is the step of mechanically treating the pressed mixture to reduce the size of the pressed mixture to yield a composition.

The term “composition” as used herein is the combination of the sweetener and ingredient treated by the processes disclosed in the present disclosure.

The teem “particulate” as used herein is a compound comprising the sweetener and ingredient, where the ingredient is at least partially embedded within the sweetener.

The term “embed” as used herein refers to the ingredient being at least partially bound within the sweetener or sweetener matrix.

The term “sweetener matrix” as used herein is a sweetener compound in which the ingredient is at least partially embedded.

The following description of the invention is intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Composition

The composition of the present disclosure is the combination of a sweetener and an ingredient prepared by a method of mixing the sweetener and the ingredient, where the sweetener comprises more than 50% by weight of the mixture, pressing the sweetener and ingredient together to form a pressed mixture, then reducing the size of the pressed mixture to yield a composition. In one embodiment, the composition comprises particulates, and a particulate comprises the ingredient at least partially embedded within the sweetener. In another embodiment, the composition comprises a sweetener matrix and an ingredient where the ingredient is at least partially embedded within the sweetener. This composition has unique properties, including a higher solubility rate, a lower apparent bulk density, and improved adherence to a food, dietary or pharmaceutical product when compared to a corresponding product prepared by blending a sweetener and ingredient having the same particle weight. The composition can be used in a variety of food, dietary or pharmaceutical products, including food products such as a meat, grain, vegetable, fruit, sauce, prepared meal, frozen food, candy, snack, chip, pre-packaged product, beverage, cereal, bar, bakery items such as bread, muffin, cookie, cake, decorating sprinkles, confectionary coating, or combinations thereof. Dietary products the composition can be used in include a dietary supplement, energy bar, beverage, powder, additive, energy drink, retort, septic ready-to-eat, ready-to-drink, or combinations thereof. The composition can also be used in a variety of pharmaceutical products including a medication, coating, solution, liquid, or combinations thereof.

### Sweetener

In an embodiment of the present disclosure, a method is disclosed to prepare a composition which comprises a sweetener and an ingredient. The sweetener can include sugars, sugar alcohols, monosaccharides, disaccharides, trisaccharides, oligosaccharides, sugar substitutes, trehalose, sucralose, stevia, saccharin, aspartame, monatin, molasses, or mixtures thereof. Monosaccharides can include fructose, glucose, galactose, and mannose. Disaccharides can include compounds such as sucrose, lactose, and maltose. Other sweeteners can include, for example, dextrose, malitol, xylitol, erythritol, polydextrose, inulin, maltodextrin, lactose, and lactitol. The sweetener can be in a variety of forms, including granulate, fine granulate, or powder fowl. Examples of common sucrose sugars include baker's sugar, beet sugar, brown sugar, cane sugar, powdered sugar, and table sugar.

### Ingredient

Examples of an ingredient that may be combined with a sweetener or a sweetener matrix in any of the embodiments described herein include, but are not limited to, a second sweetener, gums, fiber, flavors, masking agents, seasoning agents, coloring agents, aroma agents, enhancing agents, high potency agents, compounded flavors, extracts, nutrients, micronutrients, spices, herbs, oils, minerals, organic salts, inorganic salts, salt substitutes, vitamins, emulsifying agents, stabilizing agents, anti-caking agents, antioxidants, dietary supplements, starches including modified food starch, protein, or combinations thereof. By way of example and not limitation, an ingredient can also include any food ingredient that is or becomes recognized as generally recognized as safe (“GRAS”) such as those included in Title 21 of the Code of Federal Regulations (“CFR”) Parts 182, 184, and 186, in the Flavor and Extract Manufacturer's Association (“FEMA”) list, and other similar food ingredient lists in other countries. In certain embodiments, the salt substitutes may include, for example, potassium chloride, any salt substitute suitable for human consumption, or any combinations thereof. In certain embodiments, the masking agents may include any ingredient suitable for masking the bitterness or off-flavors of other compounds and suitable for human consumption, or any combination thereof. Examples of masking agents that can be used include compounds such as autolyzed yeast extracts, amino acid derivatives, monosodium glutamate, or combinations thereof. In certain embodiments, the flavor is a chemical compound or composition suitable for human consumption found to have utility in the alteration or addition of flavor or flavor characteristics of a substance, whether naturally occurring or synthetic. Flavors can include agents that provide tastes such as sweet, sour, bitter, salty, savory, spicy, umami, or combinations thereof. Flavors can also provide aromas such as alliaceous, animalic, balsamic, chemical, citrus, coffee, earthy, ethereal, fatty, floral, fruity, green, herbaceous, meaty, nutty, soapy, vegetable, waxy, wine-like and woody. Flavors can include, for example, food acidulants such as citric, lactic, malic acids and the salts of these acids or combinations thereof. High potency agent examples can include compounds such as flavor enhancers such as monosodium glutamate, or modified food starch (for texture). In certain embodiments, the coloring agents may include, for example, any natural or artificial coloring agents suitable for human consumption, or any combination thereof. The above ingredients may be used alone or in various combinations to form the composition. A variety of salts or combinations of salts suitable for human consumption can be used as the ingredient. For example, a salt can include a free flowing inorganic edible salt that is suitable for consumption, a monovalent or polyvalent salt of a polybasic food acid, or an amino acid or salt thereof. Another example of a salt can include an alkali metal or alkaline earth metal component such as sodium, potassium, magnesium, calcium, or similar ions either individually or in combination, present as the chlorides, sulfates, acetates, carbonates, gluconates or combinations thereof. Other types of salts include a chloride salt, such as sodium chloride, sea salt, potassium chloride, Fleur de Sel (or Flor de Sal), iodized salt, rock salt, or mixtures thereof. A salt can include trace amounts of other compounds, such as the trace minerals naturally present in sea salt or Fleur de Sel, or the trace amounts of iodine present in many edible salts such as iodized salt where the iodine is present in the form of sodium iodide, potassium iodide, or potassium iodate.

### Recycling Step

The sweetener and ingredient are physically combined by mixing them together where the sweetener comprises more than 50% of the mixture, pressing the mixture together under sufficient pressure to form a pressed mixture, and reducing the size of the pressed mixture to yield a composition. In an embodiment, the composition comprises particulates, and an average particulate comprises the ingredient at least partially embedded within the sweetener. The composition and particulates can be further separated into groups, depending on the composition size desired, between a nonconforming and conforming size. The conforming size can be the target size desired, whereas the nonconforming size may be oversized or undersized in relation to the conforming size. Nonconforming particulates and composition can be recycled back into the process at a variety of locations, such as in the mixing, pressing or reducing steps for further mixing, pressing or reducing. Because the composition and particulates comprise the ingredient at least partially embedded within the sweetener, they can be recycled back into the process to obtain a conforming size. In contrast, products blended by certain methods will have separation issues from handling resulting in a non-uniform blend of the sweetener and ingredient.

### Mixing Step

Mixing methods to mix the sweetener and ingredient can include placing the sweetener and ingredient into a mixing device. The sweetener and ingredient can be placed into the mixing device by means commonly known in the industry, such as by hand or by a conveyor. The mixing device can be, for example, a ribbon blender, paddle blender, plow blender, twin shell blender, double-cone blender, V-blender, single or double rotor blender, muller blender, vertical screw mixer, or similar mixing systems. The sweetener and ingredient can be placed into the mixing device for a sufficient amount of time to thoroughly mix the sweetener and ingredient. The mixing can be done either by batch mixing or a continuous process. In one batch embodiment, the sweetener and ingredient are mixed in the mixing device for about two to twenty minutes. In another embodiment, the sweetener and ingredient are mixed in the mixing device for about four to eight minutes.

### Pressing Step

Pressing methods to form the sweetener and ingredient into a pressed mixture can include compacting, flaking, pelletizing, briquetting, or other similar techniques. Once the sweetener and ingredient are mixed in the mixing step, they can be conveyed to the pressing step. For compacting or briquetting with a device such as a compactor or briquettor made by Bepex International LLC, the mixture can be force fed into the nip angle of the pressing device, such as a dual roll press with counter-rotating rolls. When the press method is flaking, such as that done with the Ludman Machine Company flaking mill, generally the mixture is gravity-fed into the press and metered or regulated to ensure a uniform distribution of the mixture. This allows a thin uniform distribution of the sweetener and ingredient mixture to be introduced into the press much like a “waterfall” of the mixture.

Pressing can be performed by applying mechanical pressure such as by compaction, flaking, pelletizing, or briquetting to the sweetener and ingredient together until the sweetener and ingredient &qui a pressed mixture such as, for example, a sheet, pellet, briquette, or other form suitable for manufacturing and/or handling. The pressing device can include, for example, a dual or twin roll press with counter rotating rolls, dual roll briquette press, dual roll compactor, flaking roll, and other similar pressing devices. In certain embodiments, the sweetener and ingredient are compacted into one or more thin sheets or flakes, or pressed into briquettes. In a preferred embodiment, the sheet is a continuous sheet having an average thickness of at least 0.005 inches. In another embodiment the sheet is a continuous sheet having an average thickness of about 0.005 inches to about 0.3 inches. In a preferred embodiment, the sweetener and ingredient are pressed together to form a pressed mixture of flakes where the flakes have an average thickness of at least 0.004 inches. In another embodiment, the flakes have an average thickness of about 0.004 inches to about 0.05 inches. In a preferred embodiment, the briquette has an average thickness of at least 0.25 inches. In another preferred embodiment, the briquette has an average thickness of about 0.25 inches to about 2.0 inches.

In one embodiment, the pressing step is run at a roll pressure of between about 0.33 and 1.333 tons per lineal inch, at a roll speed of between about 0.1 and 25 feet per second (fps). In another embodiment, the roll speed is between about 2.5 and 15 fps. In another embodiment, the pressing step is run at a roll pressure of about 1.0 tons per lineal inch at a roll speed of about 6.25 fps at ambient temperature. In one embodiment, the pressing step can be run without the addition of external heat. In another embodiment, heat or cold can be added to the pressing step. A typical particulate formed by the compaction process comprises the ingredient at least partially embedded within the sweetener, and a multitude of such particulates make up a sheet, flake, briquette, pellet or other form. Pressing the sweetener and ingredient together under pressure helps prevent separation of the sweetener and ingredient e.g., during handling, storage and/or transportation. Accordingly, the particles will impart a similar taste, color and aroma to each portion of a substrate to which the composition is applied and will exhibit other benefits such as uniform visual appearance, greater adherence, lower bulk density and faster solubility.

### Reducing Step

The pressed mixture, whether in the form of a sheet, flake, pellet, briquette, or any other form, can then be reduced, for example, by means of a roller mill, disk mill, pin mill, jaw crusher, hammer mill, jet mill, ball mill, and other similar machines to yield a composition. Reducing the size of the pressed mixture allows the product to be a composition of the particulates in a desired size for use in or on a food, dietary or pharmaceutical product or substrate. The resulting particulates of the composition comprise the ingredient at least partially embedded within the sweetener. The size of the particle can depend on the desired end-use of the final product. A smaller particle size may be desired for certain end products, whereas other applications may require a larger particulate size. In a preferred embodiment, particulate sizes may range from about 0.005 inches to 2.0 inches.

### Separating and Recycling Step

The particulates can be further separated according to size via a separation device. For instance, particulates can be further separated into groups of certain size particulates depending on the product or substrate with which it will be used, the form in which it will be sold, or the type of starting material. For example, if particulates of a target (conforming) size are desired, particulates that are oversized or undersized (nonconforming) as compared to the target size can be further separated out from the conforming sized particulates and composition. The nonconforming particulates and composition can be recycled back into the process at a variety of locations such as in the mixing, pressing, or reducing steps for further mixing, pressing or reducing. In a preferred embodiment, the amount of nonconforming material to be recycled back into the process can be in the range of 0% to 100% of the material. In other words, at least a portion of the nonconforming composition (or particulates) can be recycled back into the process in the mixing, pressing or reducing steps. In another embodiment, the separating step can occur after the pressing step. In another embodiment, the separating step can occur after the pressing step and after the reducing step. Examples of separation devices can include a sieving device, sieve shaker, vibratory screen and other similar separation devices.

The composition and particulates formed by the above method into the composition will exhibit altered or enhanced physical properties such as, for example, a high solubility rate, low bulk density, and/or improved adherence. For example, a composition in the present invention that includes a sweetener where the ingredient is at least partially embedded within the sweetener will have a solubility rate faster than a corresponding product prepared by simple blending of the sweetener and ingredient having the same average particle weight. In addition, it will have an apparent bulk density that is less than a corresponding product prepared by blending the sweetener and ingredient having the same average particle weight. Moreover, the composition will have an improved adherence to food than a corresponding product prepared by blending the sweetener and ingredient having the same average particle weight. Further, the composition and particles will impart a similar taste, aroma and color to each portion of the product to which they are applied. The particulates and composition may exhibit one or more of these altered or enhanced physical properties.

The composition of the present invention may have any variety of applications, including with a product such as a food product, a dietary product, or a pharmaceutical product. In one embodiment, the composition can be used in or on a food product such as a meat, grain, vegetable, fruit, sauce, prepared meal, frozen food, ice cream, frozen bar, frozen coating, frozen hors d′oeurvre, candy, snack, chip, pre-packaged product, beverage, dry powdered beverage, cereal, bar, bakery items such as bread, muffin, cookie, cake, decorating sprinkles, confectionary coating, or combinations thereof. In another embodiment, the composition can be used in or on a dietary product such as a dietary supplement, energy bar, beverage (such as alcohol or dairy), powder, dry powdered beverage, additive, energy drink, retort, aseptic, ready-to-eat, ready-to-drink, or combinations thereof. In another embodiment, the composition can be used in a pharmaceutical product such as a medication, coating, solution, liquid, or combinations thereof.

### EXAMPLES

### Example 1

The benefits of the composition and of the particulates that make up the composition comprising a sweetener and an ingredient at least partially embedded into the sweetener made by the present invention is evident from tests and analyses performed on the composition and examination of the composition. Samples are run on a bench scale using a Komarek Compactor as the pressing device. The roll speed for the samples is at about 14.7 feet per minute (ft/min) and at a feed rate of about 14.7 lbs/hr. The force, measured at tons per lineal inch, varied for the samples. As seen in TABLE 1 below, a number of different sweeteners and ingredients are used. In a preferred embodiment, the percent weight liquid for the ingredient is 0.5% or less. The ingredients can also be added in different forms, including but not limited to, oil, alcohol, water, encapsulated or absorbed form. The ingredients are available from Cargill, Incorporated, including encapsulated orange flavor (Cargill Flavor Systems #043-00300, 2% by weight), orange oil (Cargill Flavor Systems #040-00138, 0.2% by weight), peppermint flavor (Cargill Flavor Systems #086-03341), mixed berry flavor (Cargill Flavor Systems #126-00103), strawberry flavor (Cargill Flavor Systems #160-00492), and adsorbed orange flavor (Cargill Flavor Systems #125-02993).

TABLE 1

| | | | Force, |
|---|---|---|---|
| | | | Tons/Linear |
| | Sweetener | Ingredient | Inch |
| 1 | Erythritol | 2% encapsulated orange | 8.3 & 4.2 |
| | | flavor | |
| 2 | Sucrose, fine | 2% encapsulated orange | 8.3 & 4.2 |
| | | flavor | |
| 3 | Sucrose, granulated | encapsulated mixed berry | 8.3, 4.2 & 3.1 |
| | | flavor | |
| 4 | Sucrose, granulated | 0.2% orange oil | 10.4 |
| 5 | Sucrose, granulated | 2% encapsulated orange | 10.4 |
| | | flavor | |
| 6 | Sucrose, granulated | absorbed orange flavor | 8.3 & 4.2 |
| 7 | Sucrose, granulated | encapsulated strawberry | 10.4, 8.3 & 4.2 |
| | | flavor | |
| 8 | Sucrose, granulated | encapsulated peppermint | 8.3, 4.2 & 3.1 |
| | | flavor | |
| 9 | Sucrose, coarse | 2% encapsulated orange | 8.3 & 4.2 |
| | | flavor | |
| 10 | Rebaudioside A | 2% encapsulated orange | 8.3, 5 and 2.1 |
| | (“Reb A”) | flavor | |
| 11 | Trehalose | 2% encapsulated orange | 4.2, 3.1 & 2.1 |
| | | flavor | |
| 12 | Sucrose - 100% | | 10.4 & 4.2 |
| 13 | Erythritol - 100% | | 8.3 & 4.2 |
| 14 | Reb A - 100% | | 8.3, 5 and 2.1 |
| 15 | Trehalose - 100% | | 3.1 & 2.1 |
| 16 | Citric Acid - 100% | | 10.4, 4.2 & 3.1 |
| 17 | Inulin - 100% | | 8.3, 4.2, 2.1 |
| 18 | 60% Erythritol & | | 4.2 & 3.1 |
| | 40% Inulin | | |
| 19 | Sucrose Gran. w/ 2.8% | | 8.3 & 4.2 |
| | Reb A | | |
| 20 | 49.5% Sucrose Gran., | | 8.3, 4.2 & 3.1 |
| | 49.5% Erythritol, | | |
| | 1% Reb A | | |
| 21 | 59% Erythritol, 39% | | 8.3, 4.2 & 3.1 |
| | Inulin, 1% Reb A | | |
| 22 | 99% Trehalose, 1% | | 4.2, 3.1 & 2.1 |
| | Reb A | | |
| 23 | 50% Trehalose & | | 8.3 & 4.2 |
| | 50% KCl | | |
| 24 | SaltWise | | 8.3 |
| | 0092-1029-15-29 | | |

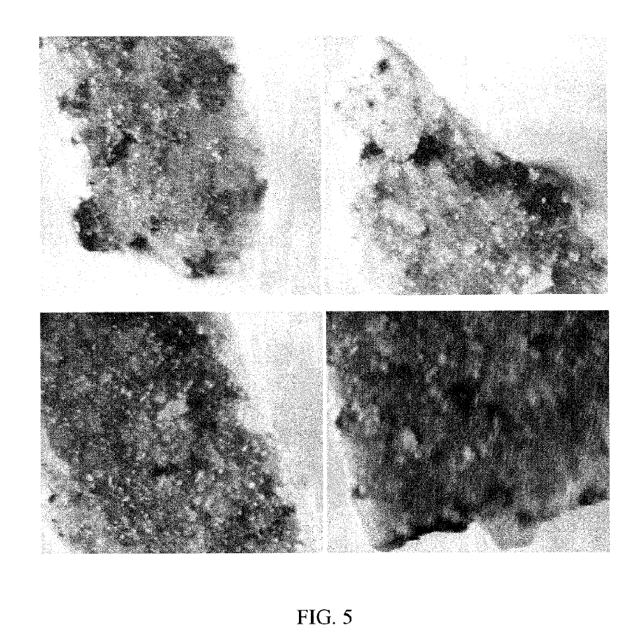

Microscopic observations of the samples such as the composition of sucrose with 2.0% encapsulated orange flavor as seen in the colored pictures in FIG. 5 show that the composition comprises particulates where the particulate comprises the ingredient at least partially embedded within the sweetener. The samples are dropped on a slide and observed under a microscope using light from various angles. The composition is different from other products where the ingredient coats the sweetener or vice versa, or where the mixture is extruded, resulting in a completely homogenous, uniform mixture of the sweetener and ingredient. The benefits of the present invention include a reduction in the loss of a compound product through preparation, handling, transportation, and even consumption of the composition.

### Example 2

Mixtures of pectin (80% by weight), sucrose (60% by weight), or maltodextrin (80% by weight) with Reb A having a purity level of 92.7% are made and run on a bench scale using a Komarek Compactor as the pressing device. The roll speed is at about 14.7 feet per minute (ft/min) and at a feed rate of about 14.7 lbs/hr. The compositions were analyzed utilizing a C18 reverse phase column on an acetonitrile:acetate buffer gradient (Phenomenex Synergi-Hydro). Three different samplings of each of the compositions were analyzed for Reb A concentration on an HPLC at 210 nm UV wavelength coupled with CAD detection.

TABLE 2

| | | Reb | | Reb A/ |
|---|---|---|---|---|
| Sample | Reb A | A/Pectin | Reb A/Sucrose | Maltodextrin |
| 1 | 90.8% | 19.0% | 34.9% | 19.0% |
| 2 | 91.4% | 18.6% | 32.9% | 18.8% |
| 3 | 91.8% | 18.90% | 32.8% | 18.9% |
| Average | 91.3% | 18.8% | 33.6% | 18.9% |
| Std Deviation (SD) | 0.48% | 0.20% | 1.2% | 0.10% |
| % relative SD | 0.52% | 1.1% | 3.6% | 0.51% |

The results in Table 2 show that in three different samplings, the Reb A concentration in the composition indicates a consistent homogeneous, uniform mixture of the sweetener and ingredient. The results further show minimal loss of Reb A after pressing (92.7% Reb A compared to average of 91.3%).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

We claim:

1. A method of preparing a composition comprising the steps of:
(a) mixing a sweetener and an ingredient, wherein the sweetener comprises more than 50% by weight of the mixture;
(b) pressing the sweetener and ingredient together to form a pressed mixture; and
(c) reducing the size of the pressed mixture to yield a composition.

2. The method of claim 1 wherein the composition comprises particulates, wherein a particulate comprises the ingredient at least partially embedded within the sweetener.

3. The method of claim 1 wherein the pressing step is performed by compaction, flaking, pelletizing or briquetting.

4. The method of claim 1 wherein the pressed mixture is foamed into a sheet, flakes, pellets, or a briquette.

5. The method of claim 4 wherein the sheet is a continuous sheet having an average thickness of at least 0.005 inches.

6. The method of claim 5 wherein the sheet is a continuous sheet having an average thickness of about 0.005 inches to about 0.3 inches.

7. The method of claim 4 wherein the briquette has an average thickness of at least 0.25 inches.

8. The method of claim 7 wherein the briquette has an average thickness of about 0.25 inches to about 2.0 inches.

9. The method of claim 4 wherein the flakes have an average thickness of at least 0.004 inches.

10. The method of claim 9 wherein the flakes have an average thickness of about 0.004 inches to about 0.05 inches.

11. The method of claim 1 wherein the sweetener comprises a natural, artificial, nutritive or non-nutritive sweetener or combinations thereof.

12. The method of claim 11 wherein the sweetener comprises sugars, sugar alcohols, monosaccharides, disaccharides, trisaccharides, oligosaccharides, trehalose, sucralose, stevia, saccharin, aspartame, monatin, dextrose, malitol, xylitol, erythritol, polydextrose, inulin, maltodextrin, lactose, lactitol, or combinations thereof.

13. The method of claim 1 wherein the ingredient comprises a flavor, seasoning agent, coloring agent, aroma agent, masking agent, enhancing agent, high potency agent, compounded flavor, extract, nutrient, micronutrient, spice, oil, herb, mineral, inorganic salt, organic salt, salt substitute, vitamin, emulsifying agent, stabilizing agent, anti-caking agent, antioxidant, dietary supplement, modified food starch, or combinations thereof.

14. The method of claim 1 wherein the ingredient is a food ingredient generally recognized as safe for consumption.

15. The method of claim 13 wherein the coloring agent is selected from natural or artificial coloring agents.

16. The method of claim 1 further comprising the step of separating the composition to yield a conforming size.

17. The method of claim 16 wherein at least a portion of a nonconforming composition is recycled back into the process in the mixing, pressing or reducing steps.

18. A composition comprising:
(a) a sweetener matrix and;
(b) an ingredient selected from the group consisting of a flavor, seasoning agent, coloring agent, aroma agent, masking agent, enhancing agent, high potency agent, compounded flavor, extract, nutrient, micronutrient, spice, oil, herb, mineral, organic salt, inorganic salt, salt substitute, vitamin, emulsifying agent, stabilizing agent, anti-caking agent, antioxidant, dietary supplement, and modified food starch, or combinations thereof, and wherein the ingredient is at least partially embedded within the sweetener matrix.

19. The composition of claim 18 wherein the sweetener matrix comprises a natural, artificial, nutritive or non-nutritive sweetener or combinations thereof.

20. The composition of claim 19 wherein the sweetener comprise sugars, sugar alcohols, monosaccharides, disaccharides, trisaccharides, oligosaccharides, trehalose, sucralose, stevia, saccharin, aspartame, monatin, dextrose, malitol, xylitol, erythritol, polydextrose, inulin, maltodextrin, lactose, lactitol, or combinations thereof.

21. The composition of claim 18 wherein the composition is used with a product, wherein the product comprises a food product, a dietary product, or a pharmaceutical product.

22. The composition of claim 21 wherein the food product comprises a meat, grain, vegetable, fruit, sauce, prepared meal, frozen food, ice cream, frozen bar, frozen coating, frozen hors d′oeurvre, candy, snack, chip, pre-packaged product, beverage, dry powdered beverage, cereal, bar, bakery items, bread, muffin, cookie, cake, decorating sprinkles, confectionary coating, or combination thereof.

23. The composition of claim 21 wherein the dietary product comprises a dietary supplement, energy bar, beverage, powder, dry powdered beverage, additive, energy drink, retort, aseptic, ready-to-eat, ready-to-drink, or combinations thereof.

24. The composition of claim 21 wherein the pharmaceutical product comprises a medication, coating, solution, liquid, or combinations thereof.
